(19) United States
(12) Patent Application Publication
(10) Pub. No.: US 2009/0180964 A1
(43) Pub. Date: Jul. 16, 2009

(54) TRANSMUCOSAL DELIVERY OF OPTICAL, SPECT, MULTIMODAL,DRUG OR BIOLOGICAL CARGO LADEN NANOPARTICLE(S) IN SMALL ANIMALS OR HUMANS

(76) Inventors: Rao Papineni, Branford, CT (US); Guizhi Li, Rochester, NY (US); John William Harder, Rochester, NY (US); Douglas Lincoln Vizard, Durham, CT (US); William E. McLaughlin, Guilford, CT (US); David L. Patton, Lebannon, PA (US)

Correspondence Address: Carestream Health, Inc., 150 Verona Street, Rochester, NY, 14608, US

(21) Appl. No.: 12/340,993

(22) Filed: Dec. 22, 2008

Related U.S. Application Data
(63) Continuation-in-part of application No. 11/165,849, filed on Jun. 24, 2005.
Application No. 11/165,849 is a continuation-in-part of application No. 11/401,343, filed on Apr. 10, 2006.
Application No. 11/401,343 is a continuation-in-part of application No. 12/221,839, filed on Aug. 7, 2008.
Application No. 12/221,839 is a continuation-in-part of application No. 12/202,681, filed on Sep. 2, 2008.
(60) Provisional application No. 61/024,621, filed on Jan. 30, 2008.
(60) Provisional application No. 61/094,147, filed on Sep. 4, 2008.

(51) Int. Cl. A61K 49/04; A61K 49/00; A61K 49/06

(52) U.S. Cl. 424/9.3; 424/9.1; 424/9.4; 424/9.6

(57) ABSTRACT

A method is taught that provides for transmucosal delivery of a biological cargo and optical molecular imaging probes to a subject animal or human. At least one biological cargo-laden nanoparticle imaging probe is provided in a form that will be absorbed via mucosal tissue. The biological cargo-laden nanoparticle imaging probe is delivered to the mucosal tissue of the animal or human. The method further may include steps of providing a support member adapted to receive the subject in an immobilized state; positioning the subject on the support member; and after the delivering of the imaging probe, imaging the immobilized subject using a multimodal imaging system.

### CROSS REFERENCE TO RELATED APPLICATIONS

Priority is claimed from provisional U.S. Patent Application Ser. Nos. (a) 61/024,621 (Docket 94735) filed on Jan. 30, 2008 by Feke et al entitled “APPARATUS AND METHOD FOR MULTI-MODAL IMAGING”; and (b) 61/094,147 (Docket 94803) filed on Sep. 4, 2008 by Papineni et al entitled “METHOD OF USE OF NEAR INFRARED FLUORESCENT IMAGING AGENTS FOR GASTRO-INTESTINAL TRACT.” The disclosure of each of these applications is incorporated by reference into this specification.

This application is a continuation in part of the following commonly assigned, co-pending U.S. patent applications, the priority of each of which is claimed and the disclosure of each of which is incorporated by reference into this specification:

Ser. No. 11/165,849 (Docket 88835CIP) filed on Jun. 24, 2005 by Bringley et al entitled “NANOPARTICLE BASED SUBSTRATE FOR IMAGE CONTRAST AGENT FABRICATION”;

Ser. No. 11/401,343 (Docket 91032) filed on Apr. 10, 2006 by Leon et al entitled “NANOGEL-BASED CONTRAST AGENTS FOR OPTICAL MOLECULAR IMAGING”;

Ser. No. 12/221,839 (Docket 94734) filed on Aug. 7, 2008 by Li et al entitled “MOLECULAR IMAGING PROBES BASED ON LOADED REACTIVE NANO-SCALE LATEX”; and

Ser. No. 12/202,681 (Docket 94731) filed on Sep. 2, 2008 by Papineni et al entitled TRANSDERMAL DELIVERY OF OPTICAL, SPECT, MULTIMODAL DRUG OR BIOLOGICAL CARGO LADEN NANOPARTICLE(S) IN SMALL ANIMALS OR HUMANS.”

### FIELD OF THE INVENTION

This invention relates generally to the transmucosal (inhaled and/or absorbed through mucosal tissue) administration into small animals or humans of compositions, such as optical, single photon emission computed tomography (SPECT), multimodal, drug or biological cargo-laden nanoparticle(s).

### BACKGROUND OF THE INVENTION

Electronic imaging systems are well known for enabling molecular imaging. An exemplary electronic imaging system 10 is shown in FIG. 1 and diagrammatically illustrated in FIG. 2. The illustrated system is the Image Station 4000 MM Multimodal Imaging System available from Carestream Health, Inc. (refer to www.carestreamhealth.com). System 10 includes a light source 12, an optical compartment 14, an optional mirror 16 within compartment 14, a lens and camera system 18, and a communication and computer control system 20 which can include a display device, for example, a computer monitor. Camera and lens system 18 can include an emission filter wheel for fluorescent imaging. Light source 12 can include an excitation filter selector for fluorescent excitation or bright field color imaging. In operation, an image of an object is captured using lens and camera system 18 which converts the light image into an electronic image, which can be digitized. The digitized image can be displayed on the display device, stored in memory, transmitted to a remote location, processed to enhance the image, and/or used to print a permanent copy of the image. U.S. Pat. No. 7,031,084 of Vizard et al, the disclosure of which is incorporated herein by reference, gives an example of an electronic imaging system suitable for lens and camera system 18.

To increase the effectiveness of these electronic imaging systems, a great deal of effort has been focused upon developing nanoparticulate probes capable of delivering imaging agents directly to the cells of interest within a test animal, human or tissue sample. These nanoparticles are also capable of carrying biological, pharmaceutical or diagnostic agents into and within living systems. These agents typically comprise drugs, therapeutics, diagnostics, biocompatibilization functionalities, contrast agents, and targeting moieties attached to or contained within a nanoparticulate carrier. Work in this field has the goals of affording imaging and therapeutic agents with such profound advantages as greater circulatory lifetimes, higher specificity, lower toxicity and greater therapeutic effectiveness. Work in the field of nanoparticulate assemblies has promised to significantly improve the treatment of cancers and other life threatening diseases and may revolutionize their clinical diagnosis and treatment.

Specific nanoparticles have been found to be nontoxic, and are capable of entry into small capillaries in the body, transport in the body to a disease site, crossing biological barriers (including but not limited to the blood-brain barrier and intestinal epithelium), absorption into cell endocytic vesicles, crossing cell membranes and transportation to the target site inside the cell. The particles in that size range are believed to be more efficiently transferred across the arterial wall compared to larger size microparticles, see Labhasetwar et al., Adv. Drug Del. Res. 24:63 (1997). Without wishing to be bound by any particular theory it is also believed that because of high surface to volume ratio, the small size is essential for successful targeting of such.

It would be desirable to produce multimodal biological targeting units or imaging probes comprising nanoparticles for use as carriers for bioconjugation and targeted delivery which are stable so that they can not only be injected in vivo, especially intravascularly, but be administered transmucosally. Further, it would be desirable that the transdermally administered nanoparticles for use as carriers be stable under physiological conditions (pH 7.4 and 137 mM NaCl). Still further, it would be desirable that such transdermally administered particles avoid detection by the immune system.

Various methods have been developed for preparing microparticles and nanoparticles to be administered transmucosally. U.S. Pat. No. 6,551,578 of Adjei et al. suggests microencapsulating a polysaccharide polymer having a selected associated medicament to be delivered to the respiratory tract of a patient to be treated in order to effect broncho-dilation or to treat a condition susceptible of treatment by inhalation, e.g., asthma, chronic obstructive pulmonary disease. U.S. Pat. No. 7,217,735 of Au et al. discloses a method for delivering microparticles and nanoparticles comprising therapeutic or apoptosis inducing agents to tissue of an animal or human patient. U.S. Published Patent Application 2005/0215475 of Ong et al. discloses the transmucosal absorption of bioactive peptides when delivered in conjunction with an absorption enhancing composition such as cationic polyamino acid. U.S. Pat. No. 6,419,949 of Gasco discloses microparticles which are prepared by dispersing in an aqueous medium at 2-4° C. a hot prepared oil/water or water/oil/water microemulsion comprising one or more lipids, a surfactant agent, a cosurfactant agent and optionally a steric stabilizer. These microparticles are suitable to the passage through the intestinal mucosa, the blood-brain barrier and the blood-cerebrospinal fluid barrier. U.S. Published Patent Application 2003/0049302 of Pauletti et al. discloses a mucoadhesive composition with an intravaginal device for vaginal delivery of effective doses of a chemotherapeutic agent or inhibitor of membrane efflux systems to the vaginal mucosa or transmucosally to the general blood circulation.

Also various methods have been developed for administering specific imaging agents via inhalation means. U.S. Pat. No. 7,198,777 of Boppart et al discloses a method of enhancing the contrast of an optical coherence tomography image of a sample by the use of microparticles, which may have a solid outer shell, an inner core, the outer shell may contain a biodegradable polymers. These microparticles may be delivered via an aerosol spray from a nebulizer, or a pressurized container that contains a suitable propellant, e.g., a gas such as carbon dioxide. U.S. Pat. No. 5,318,767 of Liversidge et al discloses an x-ray contrast composition comprising particles consisting essentially of a non-radioactive crystalline organic x-ray contrast agent having a surface modifier adsorbed on the surface in an amount sufficient to maintain an effective average particle size of less than 400 nm and suggests using inhalation as a method of administering the composition to humans and animals. U.S. Pat. No. 6,264,922 of Wood et al discloses forming an aerosol of an aqueous dispersion of insoluble diagnostic magnetic imaging agent nanoparticles having a surface modifier and administering the aerosol to the respiratory system of a mammal. U.S. Pat. No. 6,471,943 discloses transmucosal delivery of liquid or powdered aerosols comprising liposomes, polymer matrices and shells.

None of the just mentioned patents and publications provides a solution for the problem of the need for the transmucosal delivery of an optical, SPECT, multimodal, drug or biological cargo-laden nanoparticle imaging probe(s) into small animals or humans, which nanoparticles are stable under physiological conditions (pH 7.4 and 137 mM NaCl). Still further, it is desirable that the particles avoid detection by the immune system. Nor does any of the just mentioned patents and publications teach transmucosal delivery of multimodal imaging nanoparticles or the transmucosal delivery to a mouse or human for multimodal molecular imaging. It would be desirable to be able to accurately and quickly transmucosally deliver an optical, SPECT, multimodal, drug or biological cargo-laden nanoparticle(s) into small animals or humans.

In addition, for optical molecular imaging nanoparticles are needed that are less than 100 nm in size, resist protein adsorption, have convenient attachment moieties for the attachment of multimodal biological targeting units. These multimodal biological targeting units may contain emissive dyes that emit in the infrared (IR), near infrared (NIR), are capable of being detected by and enhancing X-ray imaging, being detected by and enhancing MRI imaging and being detected by and enhancing optical imaging.

Various nanoparticle probes presently are injected in vivo, especially intravascularly into both small animals for preclinical work and into humans for the diagnosis and treatment of such diseases as cancer, etc. It would be more desirable if these multimodal biological targeting units or imaging probes comprising nanoparticles could be administered transmucosally or more specifically inhaled. Inhalation and intranasal administration have been used as alternative routes of drug delivery. Inhaled drugs can be absorbed directly through the mucous membranes and epithelium of the respiratory tract, thereby minimizing initial inactivation of bioactive substances by the liver. Inhalation delivery can also provide drugs directly to therapeutic sites of action (such as the lungs or the sinuses). This mode of administration has been particularly effective for the delivery of pulmonary drugs (such as asthma medications) and peptide based drugs (usually via intranasal administration), using metered dose inhalers (MDIs). Continuous sustained administration provides better bioavailability of the drug, without peaks and troughs.

It would be desirable to be able to accurately and quickly deliver an optical, SPECT, multimodal, drug or biological cargo-laden nanoparticle(s) transmucosally to small animals or humans. Delivery could be by inhalation or other administration to and absorption by the mucosal tissue.

### SUMMARY OF THE INVENTION

The device and method of the invention will allow researchers in pharmaceutical, biotech companies, and academic setting to circumvent the invasive injection process of small animals. Particularly useful, when experiments or drug trials need tens or in some cases hundreds of small animals. Apart from the time saving process, the vital advantage is the uniformity in dose delivery when using such a system. The tail-vein injections are prone for lots of vagaries in the amounts injected.

An embodiment of the inventive method provides for transmucosal delivery of a biological cargo and optical molecular imaging probes to a subject animal or human. At least one biological cargo-laden nanoparticle imaging probe is provided in a form that will be absorbed via mucosal tissue. The biological cargo-laden nanoparticle imaging probe is delivered to the mucosal tissue of the animal or human. The imaging probe may be in the form of an aerosol delivered via nasal and oral cavities. A plurality of the imaging probes may be delivered to the subject. The imaging probe may be applied directly to the mucosal tissue in a mucous membrane location of the subject and the location may be oral, buccal, sublingual, eye, nasal, pulmonary, rectal or vaginal. The imaging probe may be laden with a material such as a drug, vaccine, biopharmaceutical, imaging contrast agent, biomolecule, or anti-infective. Several types of imaging probes are disclosed for use in accordance with the invention. The inventive method further may include steps of providing a support member adapted to receive the subject in an immobilized state; positioning the subject on the support member; and after the delivering of the imaging probe, imaging the immobilized subject using a multimodal imaging system.

### BRIEF DESCRIPTION OF THE DRAWINGS

The foregoing and other objects, features, and advantages of the invention will be apparent from the following more particular description of the embodiments of the invention, as illustrated in the accompanying drawings. The elements of the drawings are not necessarily to scale relative to each other.

FIG. 1 shows a perspective view of an exemplary electronic imaging system.

FIG. 2 shows a diagrammatic view of the electronic imaging system of FIG. 1.

FIG. 3A shows a diagrammatic side view of an imaging system suitable for use in accordance with the present invention.

FIG. 3B shows a diagrammatic front view of the imaging system of FIG. 3A.

FIG. 4 shows a perspective view of the imaging system of FIGS. 3A and 3B.

FIG. 5 schematically illustrates an embodiment of an environment for transmucosally administering bioactive materials to a mouse in accordance with the present invention.

FIG. 6A shows an X-ray image of a control mouse and an aerosol treated mouse demonstrating the experimental results of aerosol delivery of KODAK X-SIGHT 761 Imaging Agent.

FIG. 6B shows a near infrared fluorescent difference image of the control mouse and the aerosol treated mouse demonstrating the experimental results of aerosol delivery of KODAK X-SIGHT 761 Imaging Agent.

FIG. 6C shows an image where the X-ray images of FIG. 6A and the near infrared fluorescent image of FIG. 6B have been co-registered to show anatomical co-registration of the merged real time images.

FIG. 6D is an enlargement of the co-registered images of FIG. 6C.

FIG. 7A shows the experimental results of noninvasive anal delivery of KODAK X-SIGHT nanospheres to a subject mouse.

FIG. 7B shows time-lapse near infrared fluorescence images acquired after the delivery of FIG. 7A to illustrate the progression of the KODAK X-SIGHT 761 nanospheres through the subject mouse.

### DETAILED DESCRIPTION OF THE INVENTION

Reference is made to the following commonly assigned, copending U.S. patent applications, the disclosure of each of which is incorporated by reference into this specification:

(a) Ser. No. 11/221,530 (Docket 88810) filed on Sep. 9, 2005 by Vizard et al entitled “APPARATUS AND METHOD FOR MULTI-MODAL IMAGING;

(b) Ser. No. 11/400,935 by Harder et al. (Docket 91687) filed on Apr. 10, 2006 entitled “FUNCTIONALIZED POLY(ETHYLENE GLYCOL)”;

(c) Ser. No. 11/732,424 (Docket 92267) filed on Apr. 3, 2007 by Leon et al entitled “LOADED LATEX OPTICAL MOLECULAR IMAGING PROBES”;

(d) Ser. No. 11/738,558 (Docket 92737) filed Apr. 23, 2007 by Zheng et al entitled “IMAGING CONTRAST AGENTS USING NANOPARTICLES”;

(e) Ser. No. 12/196,300 filed Aug. 22, 2008 by Harder et al entitled APPARATUS AND METHOD FOR MULTI-MODAL IMAGING USING NANOPARTICLE MULTI-MODAL IMAGING PROBES, claiming priority of provisional Ser. No. 60/970,623 (Docket 93047) filed on Sep. 7, 2007 by Harder et al entitled “METHOD, APPARATUS AND PROBES FOR MULTIMODAL AND MULTISPECTRAL IMAGING”; and

(f) Ser. No. 11/930,417 by Zheng et al. (Docket 92735) filed on Oct. 31, 2007 entitled “ACTIVATABLE IMAGING PROBE USING NANOPARTICLES.”

The invention will be described in detail with particular reference to certain preferred embodiments thereof, but it will be understood that variations and modifications can be effected within the spirit and scope of the invention. Unless otherwise noted, technical terms are used according to conventional usage. Definitions of common terms in pharmacology may be found in Remington: The Science and Practice of Pharmacy, 19th Edition, published by Mack Publishing Company, 1995 (ISBN 0-912734-04-3). Transdermal delivery is discussed in particular at page 743 and pages 1577-1584. The singular forms “a,” “an,” and “the” refer to one or more than one, unless the context clearly dictates otherwise. The term “comprising” means “including.”

A “bioactive” material, composition, substance or agent is a composition which affects a biological function of a subject to which it is administered. An example of a bioactive material used to create a composition is a pharmaceutical substance, such as a drug, which is given to a subject to alter a physiological condition of the subject, such as a disease. Examples of bioactive materials that are capable of transdermal delivery include pharmaceutical compositions. As used herein, the terms “bioactive material” and/or “particles of a bioactive material” intend any compound or composition of matter which, when administered to an organism (human or nonhuman animal) induces a desired pharmacologic, immunogenic, and/or physiologic effect by local and/or systemic action. The term therefore encompasses those compounds or chemicals traditionally regarded as drugs, vaccines, and biopharmaceuticals including molecules such as proteins, peptides, hormones, nucleic acids, gene constructs and the like. More particularly, the term “bioactive material” includes compounds or compositions for use in all of the major therapeutic areas including, but not limited to, anti-infectives such as antibiotics and antiviral agents; analgesics and analgesic combinations; local and general anesthetics; anorexics; anti-arthritics; anti-asthmatic agents; anticonvulsants; antidepressants; antihistamines; anti-inflammatory agents; antinauseates; anti-migraine agents; antineoplastics; antipruritics; antipsychotics; antipyretics; antispasmodics; cardiovascular preparations (including calcium channel blockers, beta-blockers, beta-agonists and antiarrythmics); anti-hypertensives; diuretics; vasodilators; central nervous system stimulants; cough and cold preparations; decongestants; diagnostics; hormones; bone growth stimulants and bone resorption inhibitors; immunosuppressives; lipo polysaccharides, muscle relaxants; psycho stimulants; sedatives; tranquilizers; aptamers, proteins, peptides, poly arginine peptides, TAT peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including siRNA, double- and single-stranded molecules and supercoiled or condensed molecules, gene constructs, expression vectors, plasmids, antisense molecules and the like). Particles of a bioactive material, alone or in combination with other drugs or agents, are typically prepared as pharmaceutical compositions which can contain one or more added materials such as carriers, vehicles, and/or excipients.

“Carriers,” “vehicles” and “excipients” generally refer to substantially inert materials which are nontoxic and do not interact with other components of the composition in a deleterious manner. These materials can be used to increase the amount of solids in particulate pharmaceutical compositions. Examples of suitable carriers include silicone, gelatin, waxes, and like materials. Examples of normally employed “excipients,” include pharmaceutical grades of dextrose, sucrose, lactose, trehalose, mannitol, sorbitol, inositol, dextran, starch, cellulose, sodium or calcium phosphates, calcium sulfate, citric acid, tartaric acid, glycine, high molecular weight polyethylene glycols (PEG), erodible polymers (such as polylactic acid, polyglycolic acid, and copolymers thereof), and combinations thereof.

In addition, it may be desirable to include a charged lipid and/or detergent in the pharmaceutical compositions. Such materials can be used as stabilizers, anti-oxidants, or used to reduce the possibility of local irritation at the site of administration. Suitable charged lipids include, without limitation, phosphatidylcholines (lecithin), and the like. Detergents will typically be a nonionic, anionic, cationic or amphoteric surfactant. Examples of suitable surfactants include, for example, Tergitol® and Triton® surfactants (Union Carbide Chemicals and Plastics, Danbury, Conn.), polyoxyethylenesorbitans, e.g., TWEEN® surfactants (Atlas Chemical Industries, Wilmington, Del.), polyoxyethylene ethers, e.g., Brij, pharmaceutically acceptable fatty acid esters, e.g., lauryl sulfate and salts thereof (SDS), and like materials. Bioactive materials, compositions and agents also include other biomolecules, such as proteins and nucleic acids, or liposomes and other carrier vehicles that contain bioactive materials.

Mucous membranes are lubricating membrane linings on internal surfaces or an organ of an animal or human, including without limitation the alimentary, respiratory and genitourinary canals or tracts. Transmucosal delivery through absorptive mucous membranes such as the oral, buccal, sublingual, eye, nasal, pulmonary, rectal, and vaginal membranes has the advantage of being noninvasive and of bypassing hepato/gastrointestinal clearance. Aspects of the invention are described in this specification in the context of “transmucosal” delivery, unless otherwise specified. That is, the compositions, systems, and methods of the invention, unless explicitly stated otherwise, should be presumed to be applicable to transmucosal modes of delivery.

Researchers involved in the clinical testing of bioactive material compositions use tens to hundreds of small animals such as mice for these types experiments and most of these experiments involve some type of multimodal imaging of these animals. For multimodal imaging to be effective two elements are necessary. The first is a multimodal imaging system and the second is an imaging probe.

The type of imaging system described here is an example of a multimodal imaging system used by researchers to capture differing modes of imaging. This type of multimodal imaging system enables and simplifies multi-modal imaging allowing the relative movement of probes to be kinetically resolved over the time period that the animal is effectively immobilized (which can be tens of minutes). Alternatively, the same animal may be subject to repeated complete image analysis over a period of days/weeks required to assure completion of a pharmaceutical study, with the assurance that the precise anatomical frame of reference (particularly, the x-ray) may be readily reproduced upon repositioning the object animal.

Imaging modes supported by the multimodal imaging system include: x-ray imaging, bright-field imaging, dark-field imaging (including luminescence imaging, fluorescence imaging) and radioactive isotope imaging. Images acquired in these modes can be merged in various combinations for analysis. For example, an x-ray image of the object can be merged with a near IR fluorescence image of the object to provide a new image for analysis.

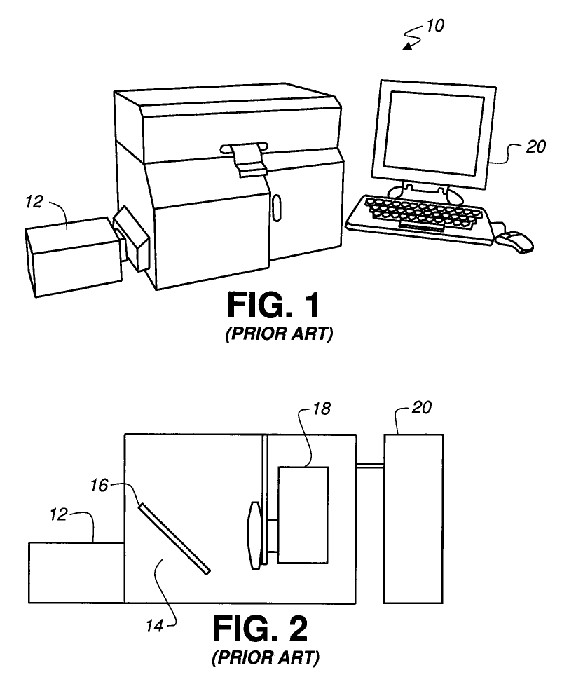

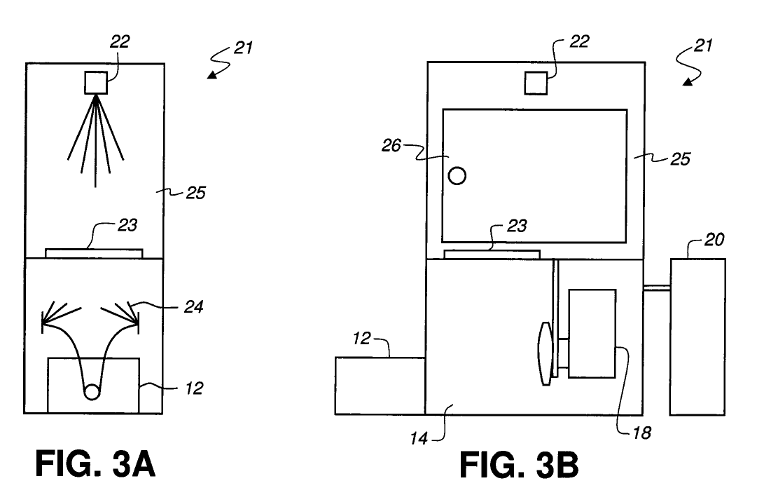

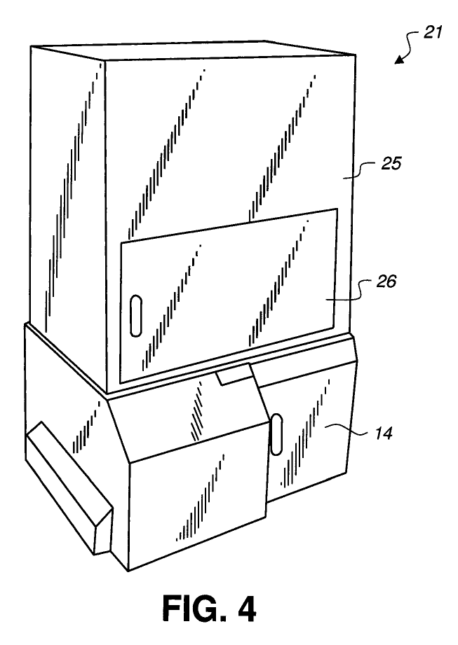

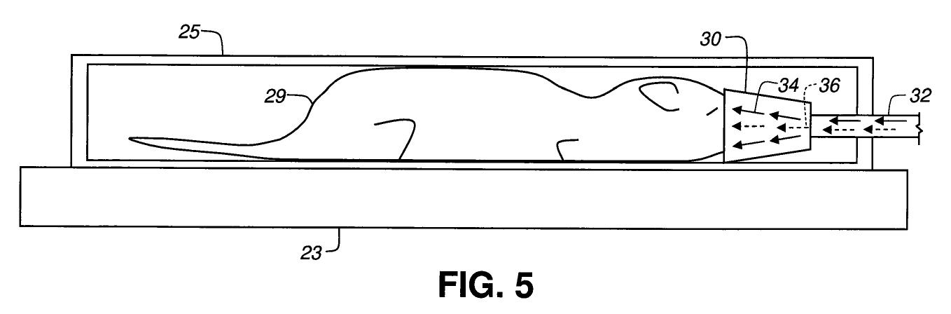

A multimodal imaging system suitable for use in accordance with the invention is illustrated in FIGS. 3A, 3B, and 4. An imaging system 21 includes the components illustrated in FIGS. 1 and 2. Also, as best shown in FIG. 3A, imaging system includes an X-ray source 22 and a sample object stage 23. Imaging system 21 further comprises epi-illumination, for example, using fiber optics 24, which directs conditioned light of appropriate wavelength and divergence toward sample object stage 23 to provide bright-field or fluorescent imaging. Sample object stage 23 is disposed within a sample environment 25, which allows access to the object being imaged. Preferably, sample environment 25 is light-tight and fitted with light-locked gas ports for environmental control. Such environmental control might be desirable for controlled x-ray imaging or for support of particular specimens. Environmental control enables practical x-ray contrast below 8 Kev (air absorption) and aids in life support for biological specimens.

Imaging system 21 further includes an access means or member 26 to provide convenient, safe and light-tight access to sample environment 25. Access means are well known to those skilled in the art and can include a door, opening, labyrinth, and the like. Additionally, sample environment 25 is preferably adapted to provide atmospheric control for sample maintenance or soft x-ray transmission (e.g., temperature/humidity/alternative gases and the like). The inventions disclosed in previously mentioned U.S. Patent Application Ser. No. 60/970,623, Ser. No. 11/221,530 and Ser. No. 61/024,621 are examples of electronic imaging systems capable of multimodal imaging and suitable for use in accordance with the present invention.

In order for multimodal imaging systems to be effective an imaging probe is needed. The “bioactive” composition previously discussed may also include various agents that enhance or improve disease diagnosis. For example, an optical, SPECT, MRI, or multimodal imaging probe may be in the form of a biological cargo-laden nanoparticle(s). The nanoparticles used for such imaging probes have many functional groups on their surfaces and are capable of conjugating with bioactive materials, such as, for example, peptides, proteins, antibodies and fragments thereof, nucleic acid, DNA or RNA and their fragments for targeting or cell penetration applications. Such nanoparticles also are able to bond chemically with drugs, imaging probes and other compounds as previously mentioned in this specification. A given nanoparticle may carry not only an imaging agent, but also one or more of the bioactive agents described, to enable the imaging agent and bioactive agents to function in a multifaceted way in accordance with the invention. The following examples describe nanoparticle imaging probes that can act as cargo carriers for such materials to provide a cargo-laden probe suitable for transmucosal delivery in accordance with the invention.

To assemble the biological, pharmaceutical or diagnostic components to a described biological cargo-laden nanoparticle used as a carrier, the components can be associated with the nanoparticle carrier through a linkage. By “associated with”, it is meant that the component is carried by the nanoparticle. The component can be dissolved and incorporated in the nanoparticle non-covalently.

Generally, any manner of forming a linkage between a biological, pharmaceutical or diagnostic component of interest and a nanoparticle used as a carrier can be utilized. This can include covalent, ionic, or hydrogen bonding of the ligand to the exogenous molecule, either directly or indirectly via a linking group. The linkage is typically formed by covalent bonding of the biological, pharmaceutical or diagnostic component to the nanoparticle used as a carrier through the formation of amide, ester or imino bonds between acid, aldehyde, hydroxy, amino, or hydrozoa groups on the respective components of the complex. Art-recognized biologically labile covalent linkages such as imino bonds and so-called “active” esters having the linkage —COOCH, —O—O— or —COOCH are preferred. The biological, pharmaceutical or diagnostic component of interest may be attached to the pre-formed nanoparticle or alternately the component of interest may be pre-attached to a polymerizeable unit and polymerized directly into the nanoparticle during the nanoparticle preparation. Hydrogen bonding, e.g., that occurring between complementary strands of nucleic acids, can also be used for linkage formation.

Preferably, imaging probes used in accordance with the invention are multimodal probes comprising a nanoparticle with one or more imaging components capable of being imaged by one or more imaging modes, including luminescence or fluorescent imaging component, X-ray, SPECT and MRI.

In the imaging probe as described in the previously mentioned U.S. patent application Ser. No. 11/401,343, the nanoparticles are in the form of a nanogel comprising a water-compatible, swollen, branched polymer network of repetitive, cross-linked, ethylenically unsaturated monomers of Formula I:

(X)m-(Y)n-(Z)o Formula I

wherein X is a water-soluble monomer containing ionic or hydrogen bonding moieties; Y is a water-soluble macromonomer containing repetitive hydrophilic units bound to a polymerizeable ethylenically unsaturated group; Z is a multifunctional cross-linking monomer; m ranges from 50-90 mol %; n ranges from 2-30 mol %; and o ranges from 1-15 mol %. The present invention also relates to a method for preparing a nanogel comprising preparing a header composition of a mixture of monomers X, Y, and Z, and a first portion of initiators in water, preparing a reactor composition of a second portion of initiators, surfactant, and water sufficient to afford a composition of 1-10% w/w of monomers X, Y, and Z; bringing the reactor composition to the polymerization temperature; holding the reactor composition at the polymerization temperature for the duration of the reaction, and adding the header composition to the reactor composition over time to form a reaction mixture, wherein the nanogel comprises a water-compatible, swollen, branched polymer network of repetitive, cross-linked, ethylenically unsaturated monomers of Formula I:

(X)m-(Y)n-(Z)o Formula I

wherein m ranges from 50-90 mol %; n ranges from 2-30 mol %; and o ranges from 1-15 mol %. For the imaging probe to be multimodal the nanoparticle making up the probe must carry two or more imaging components for example a near IR dye for fluorescent imaging and gadolinium for x-ray imaging.

In the imaging probe as described in the previously mentioned U.S. patent application Ser. No. 11/732,424, a loaded latex particle may comprise a latex material made from a mixture represented by Formula II:

(X)m-(Y)n-(Z)o-(W)p, Formula II

wherein Y is at least one monomer with at least two ethylenically unsaturated chemical functionalities; Z is at least one polyethylene glycol macromonomer with an average molecular weight of between 300 and 10,000; W is an ethylenic monomer different from X, Y, or Z; and X is at least one water insoluble, alkoxethyl containing monomer; and m, n, o, and p are weight percent ranges of each component monomer, wherein m ranges between 40-90 percent by weight, n ranges between 1-10 percent by weight, o ranges between 20-60 percent by weight, and p is up to 10 percent by weight; and wherein said particle is loaded with a fluorescent dye.

In the imaging probe as described in the previously mentioned U.S. patent application Ser. No. 11/738,558, the nanoparticles are derived from self-assembly of amphiphilic block or graft copolymers to form cross-link particles with imaging dye immobilized in the particle, more specifically the imaging dye is immobilized via covalent chemical bond in the core of the nanoparticles and alkoxy silane cross-linking results in organic/inorganic hybrid materials.

It is well known that, in the presence of a solvent or solvent mixture that is selective for on block, amphiphilic block or graft copolymers have the ability to assemble into colloidal aggregates of various morphologies. In particular, significant interest has been focused on the formation of polymeric micelles and nanoparticles from amphiphilic block or graft copolymers in aqueous media. This organized association occurs as polymer chains reorganize to minimize interactions between the insoluble hydrophobic blocks and water. The resulting nanoparticles possess cores composed of hydrophobic block segments surrounded by outer shells of hydrophilic block segments. The core-shell structures of amphiphilic micellar assemblies have been utilized as novel carrier systems in the filed of drug delivery.

The amphiphilic copolymers that are useful in the present invention have a hydrophilic water soluble component and a hydrophobic component. Useful water soluble components include poly(alkylene oxide), poly(saccharides), dextrans, and poly(2-ethyloxazolines), preferably poly(ethylene oxide). Hydrophobic components useful in the present invention include but are not limited to styrenics, acrylamides, (meth)acrylates, lactones, lactic acid, and amino acids. Preferably, the hydrophobic components derived from styrenics and (meth)acrylates containing cross-linkable alkoxy silane groups. The imaging dyes contain functional groups that can react with the cross-linkable groups of the hydrophobic component and are immobilized in the core of the nanoparticles by covalent bonding. More specifically the imaging dyes contain alkoxy silane groups. Since the imaging dyes are immobilized in the nanoparticles, the quantum efficiency is enhanced. Suitable particles are described in the previously mentioned U.S. patent application Ser. No. 11/930,417.

In the imaging probe described in the previously mentioned U.S. patent application Ser. No. 11/930,417, the nanoparticle may be in the form of an amine-modified silica nanoparticle, having a biocompatible polymer shell comprising amine functionalities. The core/shell particle has attached one or more fluorescent groups, polymer groups such as polyethylene glycol, targeting molecules, antibodies or peptides. Suitable particles are described in the previously mentioned U.S. patent application Ser. No. 11/165,949. Especially preferred are silica nanoparticles having a near infrared fluorescent core and having attached to their surface, amine groups and/or polyethylene glycol. For example the biological cargo-laden nanoparticle(s) may be a nanoparticulate imaging probe comprising an oxide core, a biocompatible polymeric shell covalently attached to the oxide core, a dye that produces emissions in response to electromagnetic radiation, a quencher that quenches the emissions of the dye, and a cleavable peptide that covalently binds the probe to a component selected from the group consisting of the dye and the quencher, such that the component is liberated from the probe when the peptide is cleaved, wherein the probe has a size of less than 100 nm and the emission of the dye molecules is quenched when the component is bound to the probe and not quenched when the component is liberated from the probe.

In multimodal imaging probes the nanoparticle has one or more imaging components capable of being imaged by one or more imaging modes such as luminescence or fluorescent imaging component, X-ray and MRI.

The luminescence or fluorescent imaging component can be a near IR dye. Fluorophores include organic, inorganic or metallic materials that luminesce with including phosphorescence, fluorescence and chemo luminescence and bioluminescence. Examples of fluorophores include organic dyes such as those belonging to the class of naphthalocyanines, phthalocyanines, porphyrins, coumarins, oxanols, flouresceins, rhodamines, cyanines, dipyrromethanes, azadipyrromethanes, squaraines, phenoxazines; metals which include gold, cadmium selenides, cadmium telerides; and proteins such as green fluorescent protein and phycobiliprotein, and chemo luminescence by oxidation of luminal, substituted benzidines, substituted carbazoles, substituted naphthols, substituted benzthiazolines, and substituted acridans.

MRI+Optical

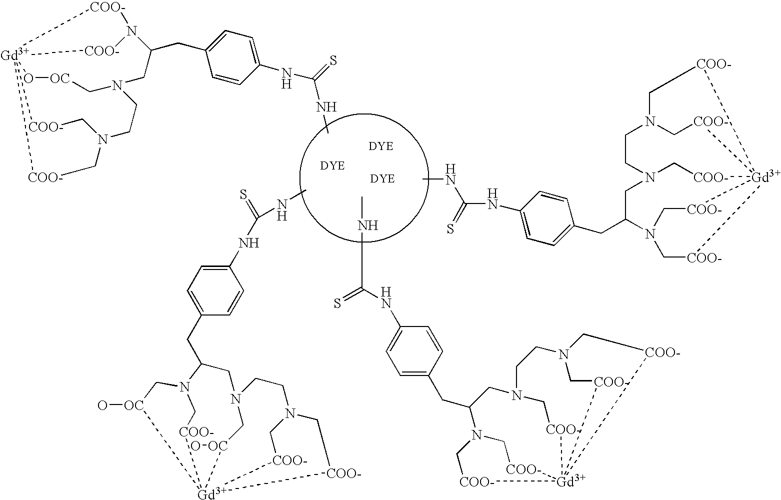

Where Dye is represented by the structure:

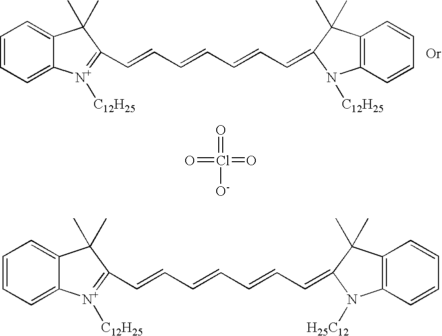

MRI Contrast Agent

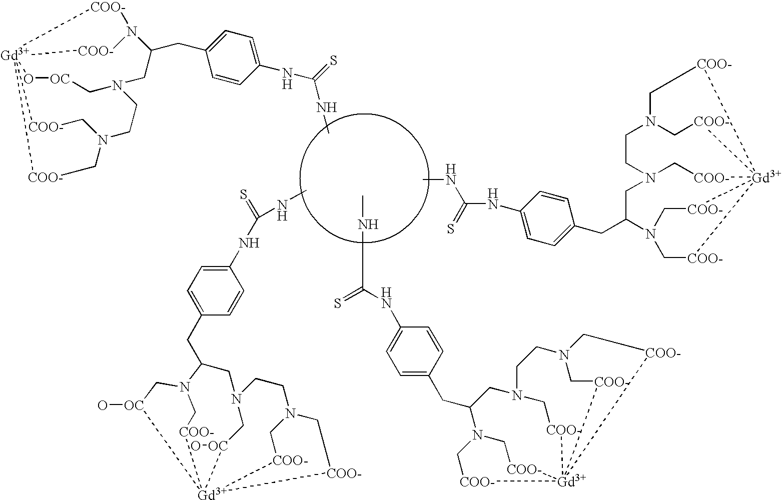

### Multimodal of Radioisotope and Dye

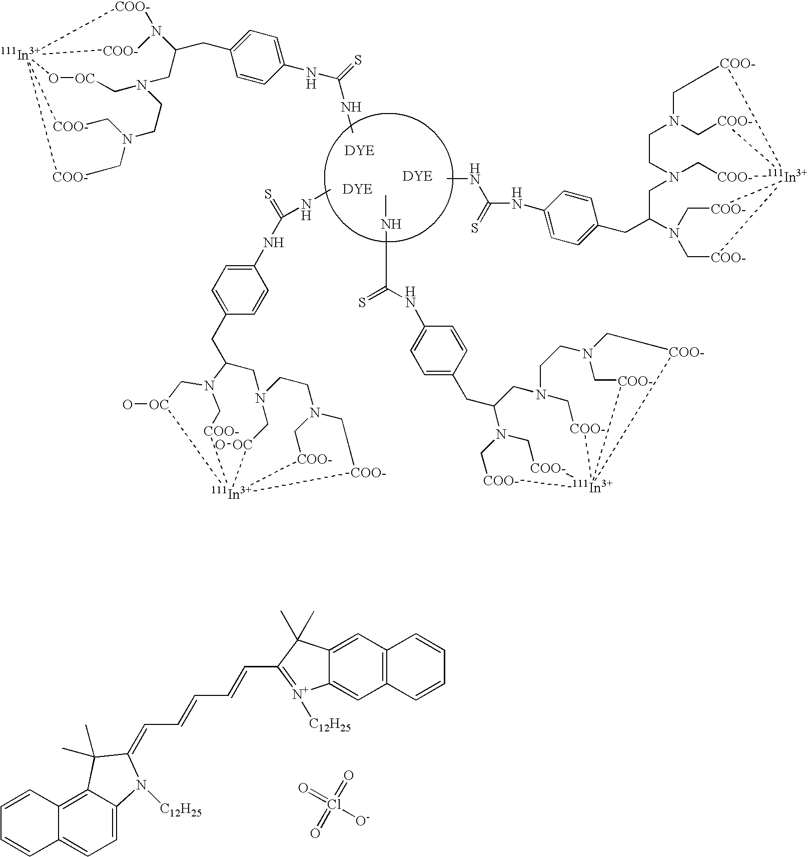

Where Dye is represented by the structure:

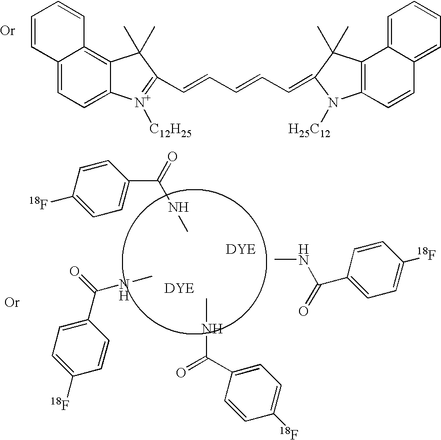

Where Dye is represented by the structure:

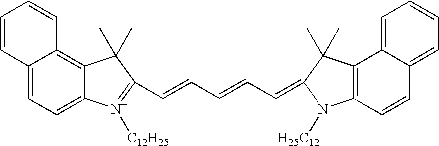

### Multimodal for X-Ray and Optical

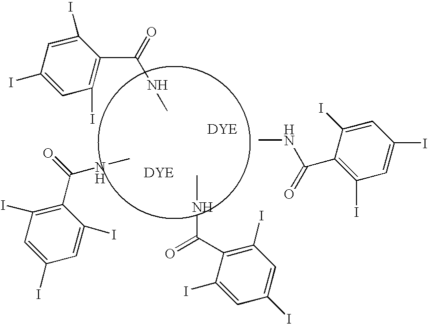

Where Dye is represented by either of the structures:

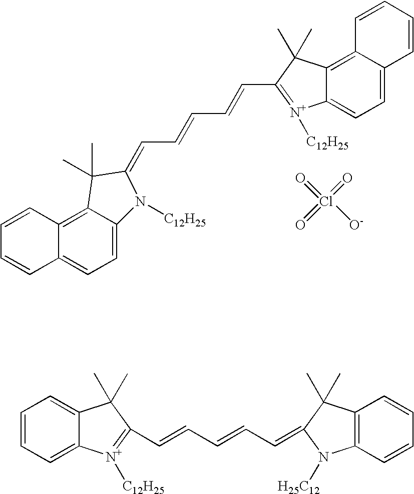

### X-Ray Contrast Agent

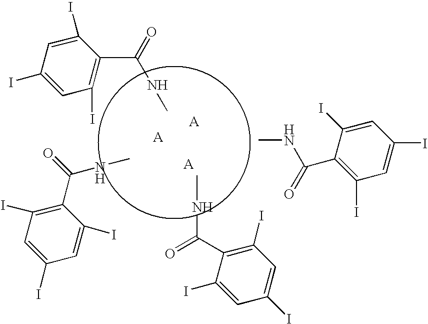

### Where A=

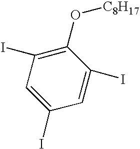

In the imaging probe as described in the previously mentioned U.S. patent application Ser. No. 12/221,839 filed Aug. 7, 2008 (Docket 94734), a biological cargo-laden nanoparticle comprising a cross-linked polymer presented in Formula III, wherein said crosslinked polymer comprises at least 45% water insoluble monomer and 1˜30 wt % monomer with reactive halo-aromatic conjugating group, and is loaded with molecular imaging agents of Formula III,

(X)m-(Y)n-(V)q-(T)o-(W)p Formula III

where m may range from 40-80 wt %, n may range from 1-10 wt %, q may range from 1-30 wt %, o may range from 10-60 wt %, and p is up to 10 wt %.
where X is a water-insoluble, alkoxyethyl-containing monomer presented in Formula IV, where R1 is methyl or hydrogen, and R2 is an alkyl or aryl group containing up to 10 carbons,

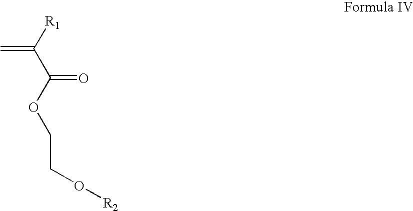

where Y is at least one monomer containing two ethylenically unsaturated chemical functionalities; W is an ethylenic monomer different from X, Y, V, or T; “V” is a polyethyleneglycol-methacrylate derivative (shown in Formula V), wherein n is greater than 1 and less than 130, preferably from 5 to 110 and CG is selected from 4-halo-3-nitrobenzoate, 2-halo-3-nitrobenzoate, 2-halo-4-nitrobenzoate, 4-halo-2-nitrobenzoate, 2-halo-5-nitrobenzoate, 3-halo-2-nitrobenzoate, 2-halonicotinate, 4-halonicotinate, 6-halonicotinate 2-haloisonicotinate, and 3-haloisonicotinate, where halo is selected from fluoro, chloro, bromo, and iodo;

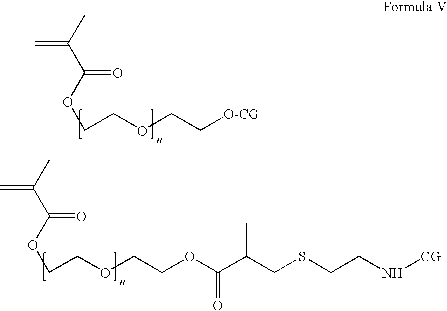

where T is a polyethyleneglycolacrylate containing macromonomer presented in Formula VI in which

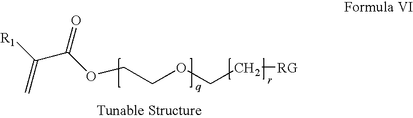

R1 is hydrogen or methyl, q is 5-220, r is 1-10, and RG is a hydrogen or functional group.

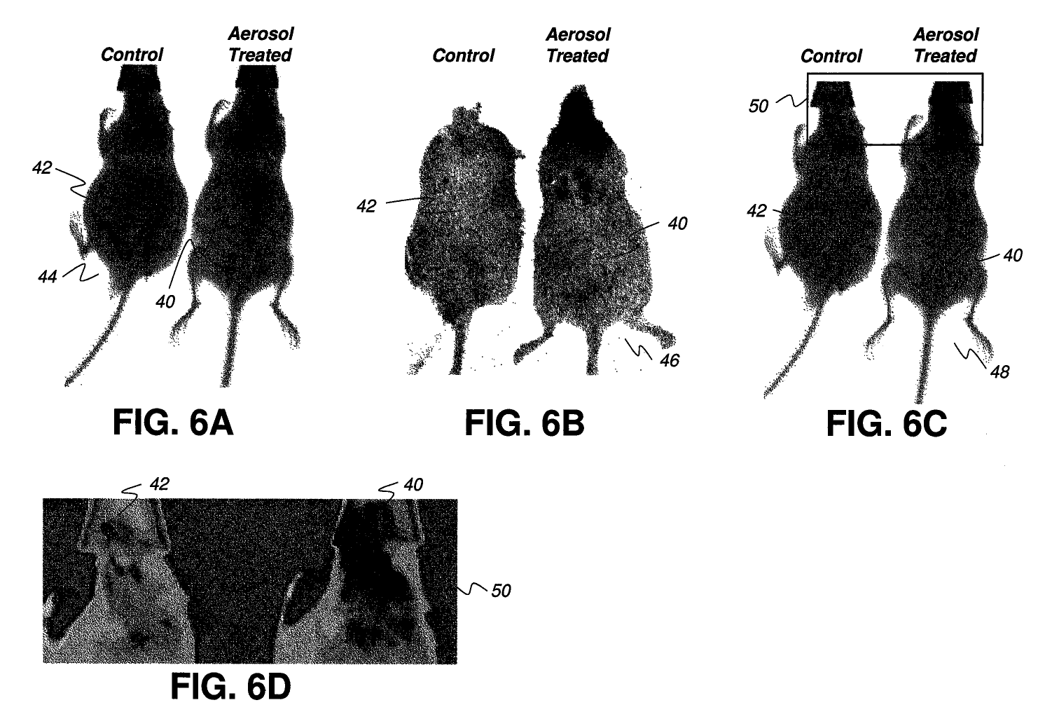

At present the primary method for administering these biological cargo-laden nanoparticle(s) is via tail-vein injections. This method of administration is both time consuming and subject to problems such as the control of the amount of bioactive material delivered. Accordingly, the present invention is directed at both an apparatus and a method for transmucosal delivery of bioactive materials (biological cargo-laden nanoparticle(s)) in a controlled active, passive or timed manner. An embodiment of an environment for transmucosally administering bioactive materials (biological cargo-laden nanoparticle(s)) is shown in FIG. 5.

Sample chamber or environment 25 and sample object stage 23 of imaging system 21 provide a location where a mouse 29 may be administered anesthesia through a respiratory device 30, such as a nose cone or mask connected to an outside source via a tube 32 which enters chamber 25 via the light-locked gas ports. The anesthesia represented by the arrows 34 sedates the mouse through out the experiment. The respiratory device 30 may also be used to transmucosally administer bioactive materials to the mouse via aerosol delivery of the biological cargo-laden nanoparticles which have been nebulized or atomized using techniques familiar to those skilled in the art, as indicated by the dotted arrows 36. The amount of the biological cargo-laden nanoparticles may be controlled via the control system 20.

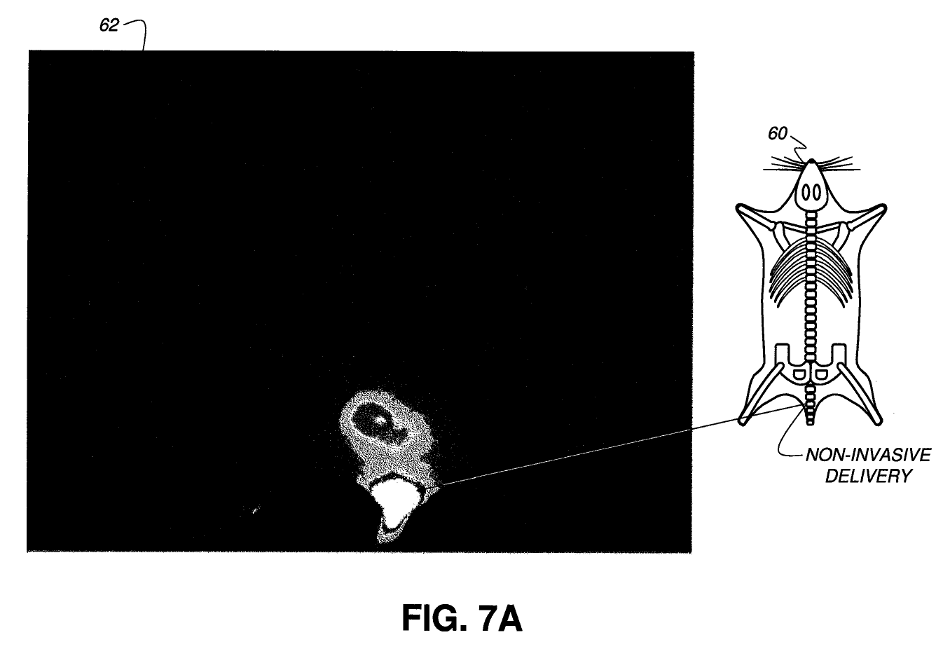

FIGS. 6A, 6B, 6C, and 6D show the experimental results of a noninvasive delivery of KODAK X-SIGHT 761 nanospheres via an aerosol to a subject mouse 40, while no KODAK X-SIGHT 761 nanospheres were delivered to a control mouse 42. The KODAK X-SIGHT 761 nanospheres were delivered to mouse 29 via respiratory device 30, causing the nanospheres to enter the nasal and oral cavities and the pulmonary system. As shown in FIG. 6A, an X-ray image 44 was taken of both subject mouse 40 and control mouse 42 with a 7 mm filter at time 0-minutes using the multimodal imaging system 21. At a time 38 minutes after capture of image 44, a near infrared fluorescent image was taken of both mice 40 and 42, and a difference image 46 was formed to highlight changes over the 38 minutes, as shown in FIG. 6B. FIG. 6C shows an image 48 which is the result of merging or co-registering of X-ray image 44 and near infrared fluorescent image 46. FIG. 6D shows an enlargement 50 of the upper part of image 48. The images captured due to signals from the KODAK X-SIGHT 761 nanospheres can readily be seen to be present in the skeletal part of the head of the subject mouse 40, while no sign of the X-Sight 761 nanospheres is seen in the control mouse 42. The experiment clearly demonstrates that the imaging agent X-Sight 761 nanospheres has successfully been transmucosally delivered to the subject mouse 40 via the mouse's nasal cavity.

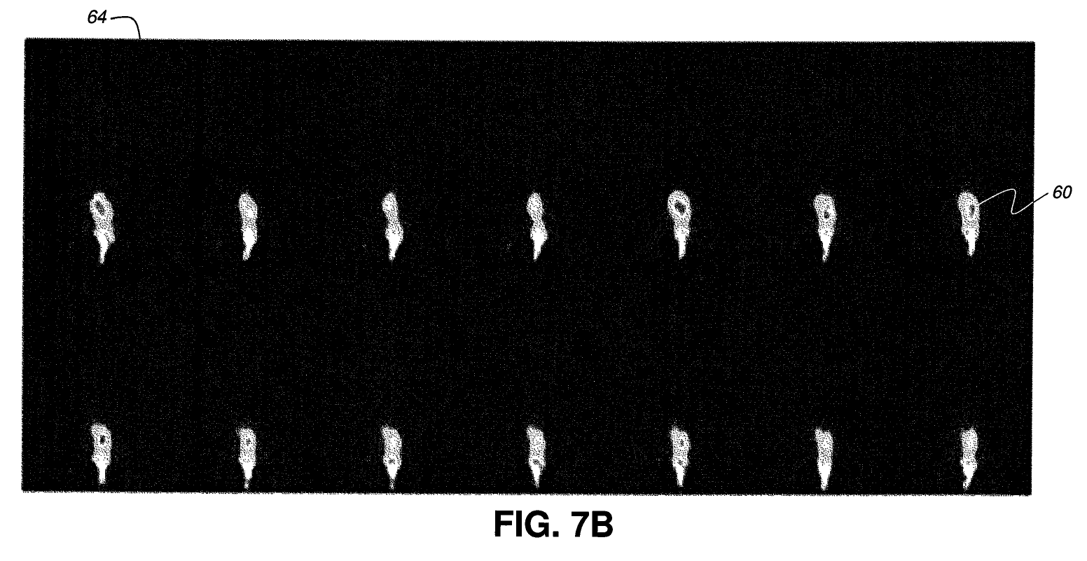

FIGS. 7A and 7B show the experimental results of a noninvasive rectal delivery of KODAK X-SIGHT 761 nanospheres to a subject mouse 60. In an experiment KODAK X-SIGHT 761 nanospheres were delivered using techniques familiar to those skilled in the art via the anal tissue to the subject mouse 60. In the case of a female mouse, the nanospheres similarly may be delivered vaginally. As shown in FIG. 7A, a near infrared fluorescent image 62 was taken of the subject mouse 60 using the multimodal imaging system 21 FIG. 7B shows a time series near infrared fluorescent images 64 of the progression of the KODAK X-SIGHT 761 nanospheres through the subject mouse 60. The near infrared fluorescent images 62 and 64 signals of the KODAK X-SIGHT 761 nanospheres can readily be seen to be present in the subject mouse 60. The experiment clearly demonstrates that the imaging agent X-Sight 761 nanospheres have successfully been delivered to the subject mouse 40 via the mouse's anal tissues. The multimodal optical imaging probes also may be delivered by tablet sublingually or in the buccal area.

In addition to using mice as test subjects, researchers also use larger animals such as rabbits, pigs, goats etc. in their experiments. When larger animals are used, the bioactive materials are administered intravascularly by injection. Again it would be very advantageous to allow researchers in pharmaceutical, biotech companies, and academic setting to circumvent the invasive injection process with the use of transmucosal delivery of these bioactive materials. The same is of course true of administering these bioactive materials to humans using the techniques described in this specification.

The invention has been described in detail with particular reference to a presently preferred embodiment, but it will be understood that variations and modifications can be effected within the spirit and scope of the invention. The presently disclosed embodiments are therefore considered in all respects to be illustrative and not restrictive. The scope of the invention is indicated by the appended claims, and all changes that come within the meaning and range of equivalents thereof are intended to be embraced therein.

### PARTS LIST

10 multimodal imaging system
12 light source
14 optical compartment
16 mirror
18 lens/camera system
20 control system
21 imaging system
22 x-ray source
23 sample object stage
24 fiber optics
25 sample environment
26 access means/member
29 mouse
30 respiratory device
32 tube
34 arrows
36 dotted arrows
40 subject mouse
42 control mouse
44 X-ray image
46 near infrared fluorescent image
48 merged image
50 enlargement
60 subject mouse
62 near infrared fluorescent image
64 series of near infrared fluorescent images

What is claimed is:

1. A method for transmucosal delivery of a biological cargo and optical molecular imaging probes to a subject animal or human, comprising:
providing at least one biological cargo-laden nanoparticle imaging probe in a form that will be absorbed via mucosal tissue; and
delivering the biological cargo-laden nanoparticle imaging probe to the mucosal tissue of the animal or human.

2. The method according to claim 1 wherein the imaging probe is in the form of an aerosol delivered via nasal and oral cavities.

3. The method according to claim 1, wherein a plurality of the imaging probes are delivered.

4. The method according to claim 1, wherein the imaging probe is applied directly to the mucosal tissue in a mucous membrane location of the subject, wherein the location is oral, buccal, sublingual, eye, nasal, pulmonary, rectal or vaginal.

5. The method according to claim 1 wherein the imaging probe is laden with a material, wherein the material is a drug, vaccine, biopharmaceutical, imaging contrast agent, biomolecule, or anti-infective.

6. The method according to claim 1 wherein the imaging probe comprises a loaded reactive latex particle comprising a cross-linked polymer presented in Formula 1, wherein said cross-linked polymer comprises at least 45% water insoluble monomer and 1˜30 wt % monomer with reactive halo-aromatic conjugating group, and is loaded with molecular imaging agents,
(X)m-(Y)n-(V)q-(T)o-(W)p Formula 1
where m may range from 40-80 wt %, , n may range from 1-10 wt %, q may range from 1-30 wt %, o may range from 10-60 wt %, and p is up to 10 wt %.
where X is a water-insoluble, alkoxyethyl-containing monomer presented in Formula 2, where R1 is methyl or hydrogen, and R2 is an alkyl or aryl group containing up to 10 carbons, 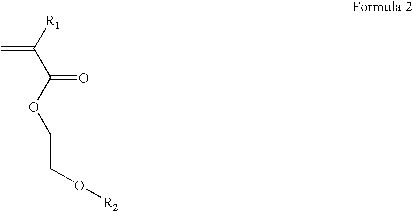
where Y is at least one monomer containing two ethylenically unsaturated chemical functionalities; W is an ethylenic monomer different from X, Y, V, or T; “V” is a polyethyleneglycol-methacrylate derivative (shown in Formula 3), wherein n is greater than 1 and less than 130, preferably from 5 to 110, and CG is selected from 4-halo-3-nitrobenzoate, 2-halo-3-nitrobenzoate, 2-halo-4-nitrobenzoate, 4-halo-2-nitrobenzoate, 2-halo-5-nitrobenzoate, 3-halo-2-nitrobenzoate, 2-halonicotinate, 4-halonicotinate, 6-halonicotinate 2-haloisonicotinate, and 3-haloisonicotinate, where halo is selected from fluoro, chloro, bromo, and iodo; 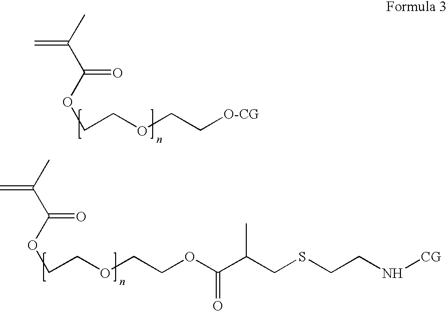
where Z is a polyethyleneglycolacrylate containing macromonomer presented in Formula 4 in which

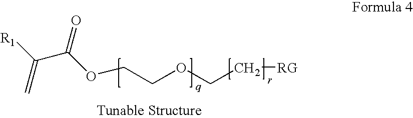

7. The method according to claim 1 wherein the imaging probe comprises a nanoparticle comprising self-assembled cross-linked, amphiphilic block copolymers and at least one immobilized dye, wherein said self-assembled, cross-linked, amphiphilic block copolymers comprise a hydrophilic block and a hydrophobic block, wherein said self-assembled, cross-linked, amphiphilic block copolymers are self-assembled to form a core of said nanoparticle comprising said hydrophobic block, wherein said hydrophobic block is derived from at least one pendant multifunctional cross-linked alkoxy silane or amino silane moiety, and an exterior of said nanoparticle comprising said hydrophilic block, and wherein said immobilized dye is immobilized in said core, and wherein said nanoparticle is not capable of dissociation when diluted in a medium.

8. The method according to claim 1 wherein the imaging probe comprises a loaded nanogel comprising a water-compatible, swollen, branched cross-linked polymer network of repetitive unsaturated monomers represented by the formula:
(X)m-(Y)n-(Z)o
wherein X is a water-soluble monomer containing ionic or hydrogen bonding moieties; Y is a water-soluble macromonomer containing repetitive hydrophilic units bound to a polymerizeable ethylenically unsaturated group; Z is a multifunctional cross-linking monomer; m ranges from 50-90 mol %; n ranges from 2-30 mol %; and o range from 1-15 mol %.

9. The method according to claim 1 wherein the imaging probe comprises a loaded latex particle comprising a latex material made from a mixture represented by formula:
(X)m-(Y)n-(Z)o-(W)p,
wherein Y is at least one monomer with at least two ethylenically unsaturated chemical functionalities; Z is at least one polyethylene glycol macromonomer with an average molecular weight of between 300 and 10,000; W is an ethylenic monomer different from X, Y, or Z; and X is at least one water insoluble, alkoxethyl containing monomer; and m, n, o, and p are weight percent ranges of each component monomer, wherein m ranges between 40-90 percent by weight, n ranges between 1-10 percent by weight, o ranges between 20-60 percent by weight, and p is up to 10 percent by weight; and wherein said particle is loaded with a fluorescent dye.

10. The method according to claim 1 wherein the imaging probe comprises an oxide core, a biocompatible polymeric shell covalently attached to the oxide core, a dye that produces emissions in response to electromagnetic radiation, a quencher that quenches the emissions of the dye, and a cleavable peptide that covalently binds the probe to a component selected from the group consisting of the dye and the quencher, such that the component is liberated from the probe when the peptide is cleaved, wherein the probe has a size of less than 100 nm and the emission of the dye molecules is quenched when the component is bound to the probe and not quenched when the component is liberated from the probe.

11. The method according to claim 1 wherein the imaging probe comprises a nanoparticle with one or more imaging components capable of being imaged by one or more imaging modes including luminescence or fluorescent imaging component, X-ray, MRI, and SPECT.

12. The method according to claim 1, further comprising:
providing a support member adapted to receive the subject in an immobilized state;
positioning the subject on the support member; and
after the delivering, imaging the immobilized subject using a multimodal imaging system.

13. The method according to claim 12 wherein the imaging is X-ray, near infrared fluorescent, magnetic resonance imaging, or SPECT.

14. The method according to claim 12 wherein the imaging probe is in the form of an aerosol delivered via nasal and oral cavities.

15. The method according to claim 12 wherein a plurality of the imaging probes are delivered.

16. The method according to claim 12 wherein the imaging probe is applied directly to the mucosal tissue in a mucous membrane location of the subject, wherein the location is oral, buccal, sublingual, eye, nasal, pulmonary, rectal or vaginal.

17. The method according to claim 12 wherein the imaging probe is laden with a material, wherein the material is a drug, vaccine, biopharmaceutical, imaging contrast agent, biomolecule, or anti-infective.

18. The method according to claim 12 wherein the imaging probe comprises a loaded reactive latex particle comprising a cross-linked polymer presented in Formula 1, wherein said cross-linked polymer comprises at least 45% water insoluble monomer and 1˜30 wt % monomer with reactive halo-aromatic conjugating group, and is loaded with molecular imaging agents,
(X)m-(Y)n-(V)q-(T)o-(W)p Formula 1
where m may range from 40-80 wt %, n may range from 1-10 wt %, q may range from 1-30 wt %, o may range from 10-60 wt %, and p is up to 10 wt %.
where X is a water-insoluble, alkoxyethyl-containing monomer presented in Formula 2, where R1 is methyl or hydrogen, and R2 is an alkyl or aryl group containing up to 10 carbons, 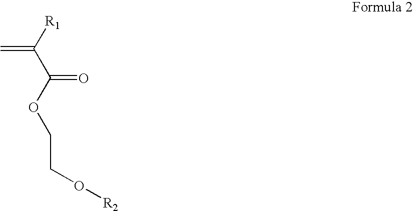
where Y is at least one monomer containing two ethylenically unsaturated chemical functionalities; W is an ethylenic monomer different from X, Y, V, or T; “V” is apolyethyleneglycol-methacrylate derivative (shown in Formula 3), wherein n is greater than 1 and less than 130, preferably from 5 to 110, and CG is selected from 4-halo-3-nitrobenzoate, 2-halo-3-nitrobenzoate, 2-halo-4-nitrobenzoate, 4-halo-2-nitrobenzoate, 2-halo-5-nitrobenzoate, 3-halo-2-nitrobenzoate, 2-halonicotinate, 4-halonicotinate, 6-halonicotinate 2-haloisonicotinate, and 3-haloisonicotinate, where halo is selected from fluoro, chloro, bromo, and iodo; 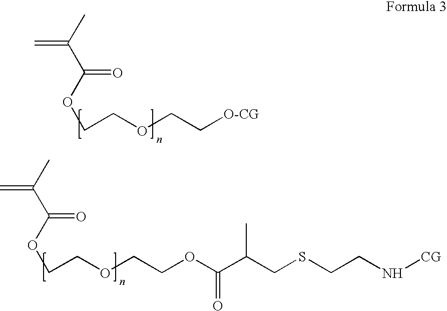
where Z is a polyethyleneglycolacrylate containing macromonomer presented in Formula 4 in which

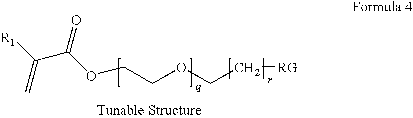

19. The method according to claim 12 wherein the imaging probe comprises a nanoparticle comprising self-assembled cross-linked, amphiphilic block copolymers and at least one immobilized dye, wherein said self-assembled, cross-linked, amphiphilic block copolymers comprise a hydrophilic block and a hydrophobic block, wherein said self-assembled, cross-linked, amphiphilic block copolymers are self-assembled to form a core of said nanoparticle comprising said hydrophobic block, wherein said hydrophobic block is derived from at least one pendant multifunctional cross-linked alkoxy silane or amino silane moiety, and an exterior of said nanoparticle comprising said hydrophilic block, and wherein said immobilized dye is immobilized in said core and wherein said nanoparticle is not capable of dissociation when diluted in a medium.

20. The method according to claim 12 wherein the imaging probe comprises a loaded nanogel comprising a water-compatible, swollen, branched cross-linked polymer network of repetitive unsaturated monomers represented by the formula:
(X)m-(Y)n-(Z)o
wherein X is a water-soluble monomer containing ionic or hydrogen bonding moieties; Y is a water-soluble macromonomer containing repetitive hydrophilic units bound to a polymerizeable ethylenically unsaturated group; Z is a multifunctional cross-linking monomer; m ranges from 50-90 mol %; n ranges from 2-30 mol %; and o range from 1-15 mol %.

21. The method according to claim 12 wherein the imaging probe comprises a loaded latex particle comprising a latex material made from a mixture represented by formula:
(X)m-(Y)n-(Z)o-(W)p,
wherein Y is at least one monomer with at least two ethylenically unsaturated chemical functionalities; Z is at least one polyethylene glycol macromonomer with an average molecular weight of between 300 and 10,000; W is an ethylenic monomer different from X, Y, or Z; and X is at least one water insoluble, alkoxethyl containing monomer; and m, n, o, and p are weight percent ranges of each component monomer, wherein m ranges between 40-90 percent by weight, n ranges between 1-10 percent by weight, o ranges between 20-60 percent by weight, and p is up to 10 percent by weight; and wherein said particle is loaded with a fluorescent dye.

22. The method according to claim 12 wherein the imaging probe comprises an oxide core, a biocompatible polymeric shell covalently attached to the oxide core, a dye that produces emissions in response to electromagnetic radiation, a quencher that quenches the emissions of the dye, and a cleavable peptide that covalently binds the probe to a component selected from the group consisting of the dye and the quencher, such that the component is liberated from the probe when the peptide is cleaved, wherein the probe has a size of less than 100 nm and the emission of the dye molecules is quenched when the component is bound to the probe and not quenched when the component is liberated from the probe.

23. The method according to claim 12 wherein the imaging probe comprises a nanoparticle with one or more imaging components capable of being imaged by one or more imaging modes including luminescence or fluorescent imaging component, X-ray, MRI, and SPECT.
